(19) United States
(12) Patent Application Publication
(10) Pub. No.: US 2007/0270659 A1
(43) Pub. Date: Nov. 22, 2007

(54) Apparatus and Method for Remotely Detecting Deception

(76) Inventor: Gary D. Giegerich, Glenside, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(21) Appl. No.: 11/381,376

(22) Filed: May 3, 2006

(51) Int. Cl. A61B 5/00

(52) U.S. Cl. 600/300

(57) ABSTRACT

A method for remotely detecting deception in information from a subject at a first location by a first interpreter positioned at a second location remote from the first location utilizing a polygraph instrument including at least one physiologic detection instrument. The method includes the steps of positioning the subject in the first location, administering the at least one physiologic detection instrument to the subject to monitor at least one physical property of the subject, prompting the subject with a stimulation, the subject responding to the stimulation with information, detecting the at least one physical property of the subject, transmitting the detected at least one physical property of the subject and the information to the second remote location and evaluating the transmitted at least one physical property and the information at the remote location. The first interpreter utilizes the polygraph instrument to evaluate the transmitted physical property and the information.

### BACKGROUND OF THE INVENTION

The present invention is directed to a mechanism and method for detecting deception in a subject or for remotely administering a lie detector test. More particularly, the present invention is directed to a method and mechanism for detecting deception in information from a subject located at a first location by a first interpreter positioned at a location remote from the first location.

Deception detection examinations, more commonly known as lie detector tests or polygraph exams, are relatively well known and commonly used deception detection methods. The lie detection tests or polygraph exams typically involve an examiner, a subject, sensory recognition equipment and a polygraph machine located at a single location that are able to determine and record changes in a subject's physical properties as the subject responds to specific questions asked by the examiner. The trained examiner observes the subject, the responses to the questions and the sensed physical properties of the subject to determine if the subject is attempting to deceive the examiner.

Polygraph examiners are relatively highly skilled and trained individuals who are in high demand. However, the polygraph examiners are in limited supply and this lack of supply coupled with the high demand results in high costs and potentially long waits for obtaining the services of the polygraph examiner and the equipment. Further, trained polygraph examiners and subjects are not typically located at a mutually convenient site and the polygraph examiner including the sensory recognition equipment and polygraph machinery must travel to a convenient site for the subject to conduct the exam. The equipment utilized for conducting the exams is also sophisticated and expensive. The complexity and cost of the equipment and the availability of the polygraph mechanisms puts their availability at a premium and results in their high cost.

It would be desirable to develop a method and mechanism to conveniently administer a deception detection or lie detector exam wherein the subject, examiner and equipment are not located at the same location. In addition, it would be desirable to develop a method and mechanism for recording and saving the deception detection or polygraph exam for replaying and additional evaluation at a later date than the originally conducted examination. Remotely conducted polygraph examinations eliminate the need for the highly trained and expensive examiner and their expensive equipment to travel to a convenient location for a polygraph subject to conduct the exam and may provide for improved accuracy in the examination as the examiner may be able to administer tests in a more uniform manner from the comfort of their own examination space.

### BRIEF SUMMARY OF THE INVENTION

Briefly stated, a preferred embodiment of the present invention is directed to a method for remotely detecting deception in information from a subject at a first location by a first interpreter positioned at a second location. The second location is remote from the first location and the first interpreter utilizes a polygraph instrument to detect the deception. The polygraph instrument includes at least one physiologic detection instrument. The method includes the steps of positioning the subject in the first location, administering the at least one physiologic detection instrument to the subject to monitor at least one physical property of the subject, prompting the subject with a stimulation, the subject responding to the stimulation with the information, detecting the at least one physical property of the subject, transmitting the detected at least one physical property of the subject and the information to the second remote location, and evaluating the transmitted at least one physical property and the information at the remote location. The first interpreter utilizes the polygraph instrument to evaluate the transmitted at least one physical property and the information for detection of deception in the subject.

In another aspect, a preferred embodiment of the present invention is directed to a lie-detection mechanism for remotely detecting deception in information given by a subject at a first location by an interpreter positioned at a second location. The second location is remote from the first location. The lie-detection mechanism includes a polygraph instrument having at least one physiologic detection instrument located at the first location. The at least one physiologic detection instrument detects at least one property of the subject at the first location. A transmission medium transmits the at least one detected property of the subject from the first location to the second location. A receiving mechanism is positioned at the second location and is in communication with the transmission medium for receiving and storing the at least one detected property of the subject. The receiving mechanism includes an output to permit the interpreter at the second location to analyze the at least one detected property to determine deception in the information given by the subject at the first location.

### BRIEF DESCRIPTION OF THE SEVERAL VIEWS OF THE DRAWINGS

The foregoing summary, as well as the following detailed description of a preferred embodiment of the invention, will be better understood when read in conjunction with the appended drawings. For the purpose of illustrating the invention, there is shown in the drawings an embodiment that is presently preferred. It should be understood, however, that the invention is not limited to the precise arrangements and instrumentalities shown. In the drawings:

FIG. 1 is a schematic diagram of an apparatus and method for remotely detecting deception in information of a preferred embodiment of the remote deception detecting method and mechanism of the present invention;

FIG. 2 is a schematic block diagram of a first location of the remote deception detecting method and mechanism shown in FIG. 1;

FIG. 3 is a schematic block diagram of the remote or second location of the remote deception detecting method and mechanism shown in FIG. 1; and

FIG. 4 is a schematic block diagram of a second interpreter interpreting a recorded examination in accordance with the remote deception detecting method and mechanism of FIG. 1.

### DETAILED DESCRIPTION OF THE INVENTION

Certain terminology is used in the following description for convenience only and is not limiting. The words “right”, “left”, “lower” and “upper” designate directions toward and away from, respectively, the geometric center of the remote deception detecting mechanism and designated parts thereof. The terminology includes the above-listed words, derivatives thereof and words of similar import.

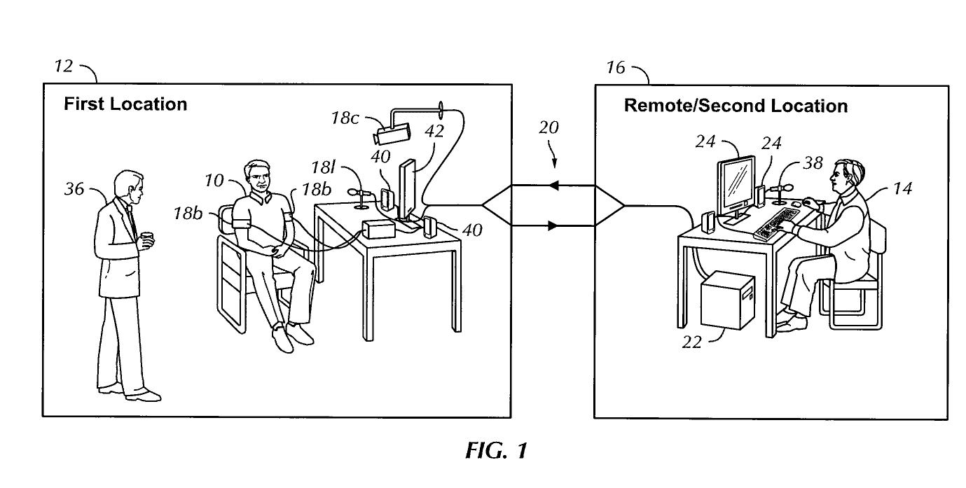

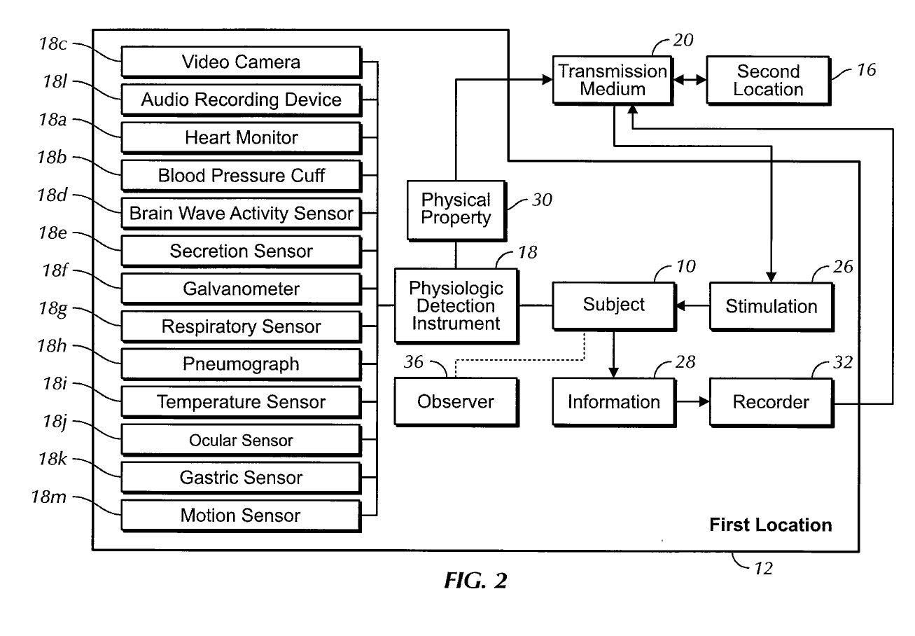

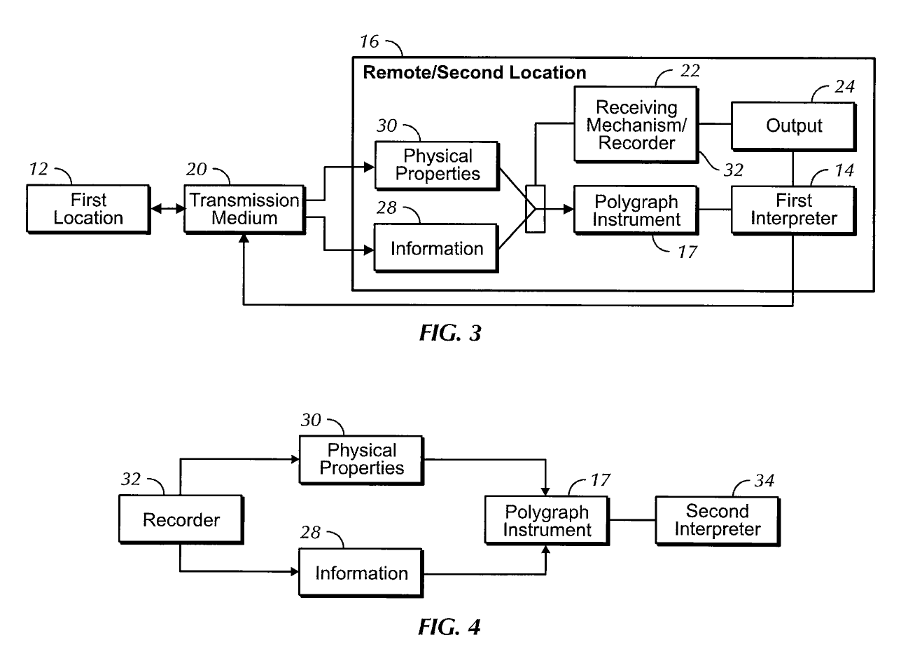

Referring to the drawings in detail, wherein like numerals indicate like elements throughout, there is shown in FIGS. 1-4 a preferred embodiment of a remote deception detecting mechanism and method for remotely detecting deception in information from a subject 10 at a first location 12 by a first interpreter 14 positioned at a second location 16 that is remote from the first location 12.

Referring to FIGS. 1-3, in the preferred embodiment, the lie-detection, deception-detection or polygraph examining equipment includes a polygraph instrument having at least one physiologic detection instrument 18 located at the first location 12. The at least one physiologic detection instrument 18 detects at least one property of the subject 10 at the first location 12. The physiologic detection instrument 18 may include, but is not limited to, one or more of a heart monitor 18a, a blood pressure cuff 18b, a video camera 18c, a brain wave activity sensor 18d, a secretion sensor 18e, a galvanometer 18f, a respiratory sensor 18g, a pneumograph 18h, a temperature sensor 18i, an ocular sensor 18j, a gastric sensor 18k, an audio recording device 18l and a motion sensor 18m. The physiologic detection instruments 18 are not limited to the above-listed instruments and may be comprised of nearly any instrument that is able to detect a property of the subject 10 during examination. The most typical physiologic detection instruments 18 utilized during the preferred remote deception detecting method include the audio recording device 18l, the video camera 18c, the pneumograph 18h, the blood pressure cuff 18b, the heart monitor 18a, the audio recording device or microphone 18l and/or the respiratory sensor 18g. The first interpreter or polygraph examiner 14 of the preferred embodiment monitors the changes in the physical properties of the subject 10 utilizing the one or more physiologic detection instruments 18 to aid in determining whether the subject 10 is attempting to deceive the examiner 14 as will be described in greater detail below.

The remote deception detecting or lie detection mechanism includes a transmission medium 20 to transmit the at least one detected property of the subject 10 from the first location 12 to the second location 16. The transmission medium 20 is preferably able to transmit the detected property of the subject 10 of the physiologic detection instruments 18 and is adaptable to receive and transmit information from the physiologic detection instruments 18. In the preferred embodiment, the transmission medium 20 is comprised of one of telephone lines, cable lines, satellite transmission equipment, cellular transmission equipment, internet transmission equipment or other like transmission equipment that is able to transmit information collected from the physiologic detection instruments 18 from the first location 12 to the second, remote location 16. The transmission medium 20 may take on many relatively well known forms to transmit the detected properties from the first location 12 to the second location 16 and is not limited to the above-listed mechanisms and methods.

The lie-detection or deception detecting mechanism also includes a receiving mechanism 22 at the second location 16 that is in communication with the transmission medium 20 for receiving and/or storing the at least one detected physiologic or physical property of the subject 10. The receiving mechanism 22 is associated with an output 24 to permit the first interpreter 14 at the second location 16 to analyze the detected physiologic property or changes in the detected property of the subject 10 to determine deception in information given by the subject 10 at the first location 12. In the preferred embodiment, the receiving mechanism 22 is comprised of a computer 22, an analog polygraph instrument (not shown), a digital polygraph instrument 22 or another like mechanism that is able to receive and/or store the transmitted properties. In addition, in the preferred embodiment, the output 24 is comprised of a video screen 24, a speaker 24, an array of light emitting diodes (not shown) and/or other like outputs that permits the first interpreter or examiner 14 at the remote location 16 to review the examination while it is occurring at the first location 12.

The receiving mechanism 22 and output 24 may be comprised of nearly any mechanism or instrument or combination of mechanisms and instruments that is able to receive data and/or information from the transmission medium 20 from the first location 12 and store and output the information to the first interpreter 14 at the second location 16. The preferred receiving mechanism 22 is comprised of the computer 22 which is able to receive and store the information from the transmission medium 20 and communicate with the output 24, which is preferably comprised of at least a computer monitor or screen 24 and speakers 24 for outputting the information to the first interpreter or polygraph examiner 14. The receiving mechanism 22 and output 24 are not limited to the above-described items and apparatus and may be comprised of nearly any mechanism that is able to receive, store and output the received information from the first location 12 from the transmission medium 20 at the second location 16 and communicate the information to the first interpreter or examiner 14.

In operation, the subject 10 is positioned at the first location 12, at least one of the physiologic detection instruments 18 is administered to the subject 10 to monitor at least one physical property of the subject 10, the subject 10 is prompted with a stimulation 26, the subject 10 responds to the stimulation 26 with information 28, at least one physical property of the subject 10 is detected using one of the physiologic detection instruments 18, the detected physical property of the subject 10 and the information 28 are transmitted to the second location 16 and the at least one transmitted physical property 30 and the information 28 are evaluated by the first interpreter 14 at the second or remote location 16. The first interpreter 14 preferably utilizes the polygraph instrument to evaluate the transmitted physical property 30 and the information 28 to detect deception in the subject 10.

Referring to FIGS. 1-4, in the preferred embodiment, the at least one detected physical property 30 of the subject 10 is constantly recorded by a recorder 32 at either or both of the first or second locations 12, 16 or at an additional location (not shown) that is in communication with the transmission medium 20. The physical properties 30 recorded from the subject 10, information 28 given by the subject 10 and the stimulation 26 administered to the subject 10 are each recorded by the recorder 32 during the course of the examination for later use. In addition, the changes in the physical properties 30, information 28 and stimulation 26 are recorded. At a later time, the recorded information in the recorder 32 may be outputted for examination by a second interpreter 34. Accordingly, the results of the examination may be verified, double-checked or otherwise reviewed by the second trained polygraph examiner or interpreter 34. The second evaluation or examination by the second interpreter 34 is preferably conducted after the initial review by the first interpreter 14 and may occur a significant amount of time, even years after the initial examination by the first interpreter 14. The second interpreter 34 preferably, independently determines based upon the recorded information 28, stimulation 26 and physical properties 30 whether the subject 10 is attempting to deceive during the examination.

In the preferred embodiment, the first and second interpreters 14, 34 are comprised of a trained examiner, a polygraph examiner, a forensic psychophysiologist, a law enforcement officer, a security firm, a polygraph regulator or another trained interpreter or examiner that is able to evaluate the recorded physical properties 30, information 28 and stimulation 26 to determine if the subject 10 is attempting to deceive, otherwise indicating that the subject 10 is lying. The first and second interpreters 14, 34 are not limited to the above-listed individuals with the specialties that are generally understood by one having ordinary skill in the art and may be comprised of nearly any individual that is able to interpret the information 28, stimulation 26, recorded physical properties 30 or other information to determine whether the subject 10 is attempting to deceive.

In the preferred utilization of the deception detecting mechanism and method, prior to conducting the examination, the identity of the subject 10 is verified, a pretest of the subject 10 is conducted and the subject 10 is examined for evidence of countermeasures at the first location 12. An observer 36, preferably located at the first location 12, is preferably able to perform these tasks prior to the examination. The observer 36 is typically not as highly trained or skilled as the first and second interpreters 14, 34 and may be nearly any individual who is able to setup the examination apparatus at the first location 12 and perform the typical tasks of the observer 36. For example, the observer 36 may merely verify the identity of the subject 10, apply the physiologic detection instruments 18 to the subject and generally observe the subject 10 during the examination process. However, the observer 36 is not limited to being a less skilled individual than the examiners 14, 34 and may be a highly trained polygraph examiner. For example, the observer 36 is typically at least moderately trained if the observer 36 conducts the pre-test of the subject 10 prior to the examination or is independently conducting a deception detection or polygraph examination at the first location 12 as the first interpreter 14 is also conducting the examination at the second location 16. For example, utilizing the observer 36 as an additional examiner may aid in verification of the results of the first interpreter or examiner 14 and may increase the acceptance or reliability of the results of the examination, depending upon the ultimate determination of the observer 36, the first examiner 14 or the first interpreter 14.

The observer 36 typically observes the examination of the subject 10 and also aids in directing the video camera 18c on to the subject, positioning the audio recording device or microphone 18l approximate the subject 10, attaching the heart rate monitor 18a to the subject 10, attaching the blood pressure cuff 18b to the subject 10, attaching the brain wave utility sensor 18d to the subject 10, attaching the secretion sensor 18e to the subject 10, attaching the galvanometer 18f to the subject 10, attaching the respiratory sensor 18g on or proximate to the subject 10, attaching the pneumograph 18h to the subject 10, attaching a temperature sensor 18i to the subject 10, attaching the ocular sensor 18j to the subject 10, attaching the gastric sensor 18k to the subject 10 and mounting the motion sensor 18m on or proximate to the subject 10. The observer 36 is not limited to these roles in the examination process at the first location 12 and may have an expanded or more limited role in preparing the subject 10 in first location 12 or for generally preparing the first location 12 for the examination.

In the preferred embodiment, the stimulation 26 is preferably comprised of a series of oral or written questions. The oral questions are preferably provided by the first interpreter 14 by speaking into a microphone 38 at the second location 16 and the oral questions are transmitted by the transmission medium 20 to the first location 12 where the oral questions are output by a speaker 40 to the subject 10. Alternately, the stimulation 26 may be comprised of written questions, which are prepared by the first interpreter 14 and given to the subject 10 by the observer 36 or presented to the subject 10 during the examination on a monitor 42 located at the first location 12. The oral or written questions 26 are answered by the subject 10, thereby providing the information 28. During the time that the subject 10 is answering the oral or written questions 26, the physical properties of the subject 10 are being constantly detected by the physiologic detection instruments 18 and the detected physical properties 30 and information 28 provided by the subject 10 is constantly being transmitted to the second location 16 through the transmission medium 20. The first interpreter 14 preferably receives the information 28 and physical properties 30 on a computer monitor 24 and speakers 24 at the second location 16. The provided information 28 of the subject 10 and physical properties 30 are preferably recorded by the recorder 32 and/or in receiving mechanism 22 for later use.

Alternatively, the observer 36 may provide the oral or written questions or alternate stimulation 26 directly to the subject 10 at the first location 12. This method may most effectively be applied when the observer 36 is an at least a moderately skilled and trained individual who has performed the pre-test on the subject 10 but this is not a limiting element and the stimulation 26 may be provided in nearly any manner to the subject 10, without departing from the broad inventive concept of the present application. The preferred oral or written questions or other stimulation 26 may also be developed and provided as a standard or by the second interpreter 34 to stimulate or prompt the subject 10.

In the preferred embodiment, the first interpreter 14 is able to transmit information from the second location 16 to the observer 36 at the first location 12. For example, the first interpreter 14 at the second location 16 may be in cellular or other communication exclusively with the observer 36 without knowledge of the subject 10 or may be in communication with the observer 36 with the full knowledge of the subject 10 through oral or visual mechanisms including the speakers 40 and display screen 42. Accordingly, during the examination process, the first interpreter 14 may provide directions to the observer 36 to alter or manipulate the physiologic detection instruments 18 or other of the equipment or the subject 10 at the first location 12.

In the preferred embodiment, the method may be adapted such that the subject 10 is stimulated by a stimulation 26 comprised of an oral or written question presented in a first language and the subject 10 provides the information 28 in the first language. The information 28 is then translated from the first language to a second language that the first interpreter 14 is familiar with at the second location 16. The information 28 may be translated by the observer 36 or by the detection equipment. For example, the computer 22 at the second location 16 may be adapted to have the ability to translate written or oral information 28 transmitted through the transmission medium 20 from the first location 12 at the second location 16. In addition, the computer 22 may also be adapted to translate oral or written commands from the first interpreter 14 at the second location 16 for transmission over the transmission medium 20 and stimulation of the subject 10 in their own language at the first location 12. Alternatively, the observer 36 may perform all of the written or oral translations during the examination.

Preferably, through use of the deception detecting mechanism and method, the first interpreter 14 at the second location 16 is able to review the stimulation 26, information 28 and physical properties 30 at the second location 16 and provide an indication of deception of the subject 10 based upon the examination. Positioning the first interpreter 14 and the interpreter's relatively expensive equipment at the second location 16 provides the benefit that the highly trained and skilled first interpreter 14 and the generally high-priced equipment of the first interpreter 14 may be located at the second location 16 and examinations may be performed on subject's 10 that are located at a convenient first location 12. In addition, the examiner or interpreter 14 may conduct several examinations in a single day or over a relatively short period of time at the convenient second or remote location 16 without the necessity to travel to the location of the subject 10 or, potentially, to the numerous distanced locations of various subjects 10. In addition, the utilization of the preferred observer 36 permits the interpreter 14 to provide some control over the examination environment at the first location 12 to limit countermeasure techniques that may be utilized by the subject 10, for example, placing a tack in the subject's shoe 10 to alter physical responses to the stimulation 26 during the examination or other countermeasures that are generally well known to those having ordinary skill in the art. In addition, the observer 36 is able to verify the identity of the subject 10 such that the proper subject 10 does not send an associate to take the deception examination.

It will be appreciated by those skilled in the art that changes could be made to the embodiment described above without departing from the broad inventive concept thereof. It is understood, therefore, that this invention is not limited to the particular embodiment disclosed, but it is intended to cover modifications within the spirit and scope of the present invention as defined by the appended claims.

We claim:

1. A method for remotely detecting deception in information from a subject at a first location by a first interpreter positioned at a second location remote from the first location utilizing a polygraph instrument including at least one physiologic detection instrument, the method comprising the steps of:
a) positioning the subject in the first location;
b) administering the at least one physiologic detection instrument to the subject to monitor at least one physical property of the subject;
c) prompting the subject with a stimulation, the subject responding to the stimulation with the information;
d) detecting the at least one physical property of the subject;
e) transmitting the detected at least one physical property of the subject and the information to the second remote location; and
f) evaluating the transmitted at least one physical property and the information at the remote location, the first interpreter utilizing the polygraph instrument to evaluate the transmitted at least one physical property and the information for detection of deception in the subject.

2. The method for remotely detecting deception of claim 1 comprising the further step of:
g) recording the transmitted and detected at least one physical property and the information of steps (d) and (e) at one of the first and second locations; and
h) outputting the recorded at least one physical property and the information following step (f) for evaluation by a second interpreter.

3. The method for remotely detecting deception of claim 2 wherein step (h) occurs at a time subsequent to step (f).

4. The method for detecting deception of claim 2 comprising the further step of:
i) outputting the recorded at least one physical property and the information at a later time to a second interpreter such that the second interpreter is able to evaluate the recorded at least one physical property and the information for detection of deception in the subject.

5. The method for detecting deception of claim 4 wherein the first and second interpreters are comprised of at least one of a trained examiner, a polygraph examiner, a forensic psychophysiologist, a law enforcement officer, a security firm and a polygraph regulator.

6. The method for detecting deception of claim 1 comprising the further steps of:
g) verifying the identity of the subject prior to step (a);
h) conducting a pretest of the subject prior to step (a); and
i) examining the subject for evidence of countermeasures at the first location prior to step (a), wherein steps (g), (h) and (i) are performed by an observer located at the first location.

7. The method for detecting deception of claim 1 wherein steps (a) and (b) are performed by an observer at the first location, the observer monitoring the subject during steps (c) and (d).

8. The method for detecting deception of claim 1 wherein an observer as part of step (b) performs at least one of the steps of:
b1) directing a video camera onto the subject;
b2) positioning an audio recording device proximate the subject;
b3) attaching a heart monitor to the subject;
b4) attaching a blood pressure cuff to the subject;
b5) attaching a brain wave activity sensor to the subject;
b6) attaching a secretion sensor to the subject;
b7) attaching a galvanometer to the subject.
b8) attaching a respiratory sensor one of to or proximate to the subject;
b9) attaching a pneumograph to the subject;
b10) attaching a temperature sensor one of to or proximate to the subject;
b11) attaching an ocular sensor to the subject;
b12) attaching a gastric sensor to the subject; and
b13) attaching a motion sensor one of or proximate to the subject.

9. The method for detecting deception of claim 1 wherein in step (c) the subject is prompted with a series of one of oral and written questions.

10. The method of detecting deception of claim 9 wherein the first interpreter asks the oral question through a microphone located at the first location and visually and auditorily receives the information and the detected at least one physical property of the subject at the second location.

11. The method of detecting deception of claim 1 wherein an observer at the first location prompts the subject in step (c).

12. The method of detecting deception of claim 1 comprising the further step of:
g) transmitting information from the first interpreter at the second location to an observer at the first location.

13. The method of detecting deception of claim 1 comprising the further step of:
g) providing an indication of deception related to the information following step (f).

14. The method of detecting deception of claim 1 wherein an observer prepares the at least one physiologic detection instrument prior to step (b) and reviews the subject and the at least one physiologic detection instrument during steps (c)-(e).

15. The method of detecting deception of claim 1 wherein the subject is prompted in step (c) by a question presented in a first language, the subject providing the information in the first language, the information being translated from the first language to a second language in one of steps (e) and (f).

16. The method of detecting deception of claim 1 comprising the further step of:
g) transmitting the stimulation from the first interpreter at the second location to the first location prior to step (c), the stimulation comprised of a question in a second language; and
h) translating the stimulation in the second language to a first language, the subject being familiar with the first language and being unfamiliar with the second language.

17. The method of detecting deception of claim 16 wherein step (h) is performed by an observer located at the first location.

18. A lie-detection mechanism for remotely detecting deception in information given by a subject at a first location by an interpreter positioned at a second location remote from the first location, the lie-detection mechanism comprising:
a polygraph instrument including at least one physiologic detection instrument located at the first location, the at least one physiologic detection instrument detecting at least one property of the subject at the first location;
a transmission medium to transmit the at least one detected property of the subject from the first location to the second location; and
a receiving mechanism at the second location and in communication with the transmission medium for receiving and storing the at least one detected property of the subject, the receiving mechanism including an output to permit the interpreter at the second location to analyze the at least one detected property to determine deception in the information given by the subject at the first location.

19. The lie-detection mechanism of claim 18 wherein the at least one physiologic detection instrument includes at least one of a heart monitor, a blood pressure cuff, a video camera, a brain wave activity sensor, a secretion sensor, a galvanometer, a respiratory sensor, a pneumograph, a temperature sensor, an ocular sensor, a gastric sensor, an audio recording device and a motion sensor.

20. The lie-detection mechanism of claim 18 wherein the transmission medium is comprised of at least one of telephone lines, cable lines, satellite transmission equipment, cellular transmission equipment and internet transmission equipment.

21. The lie-detection mechanism of claim 18 wherein the receiving mechanism is comprised of at least one of a computer, an analog polygraph instrument, a video screen, a digital polygraph instrument, a speaker and an array of light emitting diodes.
